(19) United States
(12) Patent Application Publication
(10) Pub. No.: US 2026/0168008 A1
(43) Pub. Date: Jun. 18, 2026

(54) MUTANT GENE DETECTION METHOD

(71) Applicant: HITACHI HIGH-TECH CORPORATION, Tokyo (JP)

(72) Inventors: Hirokazu KATO, Tokyo (JP); Motohiro YAMAZAKI, Tokyo (JP); Isao HARAURA, Tokyo (JP); Noriyuki SUMIDA, Tokyo (JP)

(21) Appl. No.: 19/129,340

(22) PCT Filed: Nov. 22, 2022
(86) PCT No.: PCT/JP2022/043106

§ 371 Date: May 13, 2025

(51) Int. Cl. C12Q 1/6818; C12Q 1/683

(52) U.S. Cl. CPC C12Q 1/6818; C12Q 1/683

(57) ABSTRACT

The present disclosure aims to provide a technique capable of accurately detecting a mutant gene and a mutation rate using capillary electrophoresis. A mutant gene detection method according to the present disclosure classifies signal peaks included in a detected signal into a first group that is lower than a first threshold and a second group that is not lower than the first threshold, increases an injection voltage until the signal peak belonging to the first group becomes higher than or equal to the first threshold, and reduces the injection voltage after the increase in the injection voltage until the signal peak belonging to the second group becomes lower than or equal to a second threshold which is higher than the first threshold (see FIG. 7).

### TECHNICAL FIELD

The present disclosure relates to a method of detecting a mutant gene.

### BACKGROUND ART

DNA analysis using electrophoresis includes fragment analysis and sequence analysis. Examples of the fragment analysis include personal identification, microsatellite instability (MSI) analysis, and multiplex ligation-dependent probe amplification (MLPA).

As a method of detecting a mutant gene using MLPA, there is methylation-specific MLPA (MS-MLPA) (Nonpatent Literature 1).

In MS-MLPA, two adjacent probes that specifically bind (hybridize) to a target gene (region) are used. To each of the probes, a common sequence that enables PCR amplification with a universal primer is bound. The probes are designed to provide different amplified fragment lengths. The two adjacent probes that hybridized to a target gene sequence are joined together by a ligase. After the hybridization, separate tubes are used for copy number analysis and methylation analysis, and at the same time as the ligation reaction, the tubes are treated with a methylation-sensitive restriction enzyme Hha1, and a PCR is performed. A probe in an unmethylated region is cleaved by the restriction enzyme and is therefore not amplified by the PCR. A probe in a methylated region is not cleaved and is amplified by the PCR. Detected signals are obtained by electrophoresisg an obtained DNA fragment using a capillary electrophoresis apparatus. Based on a difference between positions of peaks of the detected signals, it is possible to identify an unmethylated cell (normal cell) and a methylated cell (cancer cell).

The following Patent Literature 1 describes DNA analysis using capillary electrophoresis. In the literature, when a detected signal obtained by the electrophoresis is saturated (when the detected signal exceeds a recordable upper limit), a flag that prompts a user to adjust an injection parameter is output (0165 of the literature). Furthermore, a signal-to-noise ratio of an optical signal is calculated using a median value of a signal peak and is compared with noise estimated from a non-peak region (0166 of the literature).

### CITATION LIST

### Patent Literature

### Patent Literature 1: US2020/0003728A1

### Nonpatent Literature

Nonpatent Literature 1: https://www.falco-genetics.com/salsa/principle.html

### SUMMARY OF INVENTION

### Technical Problem

Since the amount of a mutant gene is very small, a detected signal from the mutant gene is very weak, and the signal strength may fall below a detectable lower limit. In this case, an experimenter needs to increase an injection voltage and a sample concentration and perform the electrophoresis again. However, when the injection voltage and the sample concentration are increased, the detected signal becomes saturated and a mutation rate cannot be calculated. This is due to the fact that a signal level of a saturated peak cannot be identified, and therefore the ratio of a signal peak level derived from a mutant type to a signal peak level derived from a wild type cannot be calculated.

In such a conventional technique as described in Patent Literature 1, all of signal peaks need to be higher than or equal to the detectable lower limit and need to be unsaturated, and if any of the conditions is not satisfied, the injection voltage and the like are adjusted and the electrophoresis is performed again until both of the conditions are satisfied. Therefore, from a DNA sample, such as a mutant gene, which causes a very weak signal peak and a normal signal peak, it is considered difficult to detect them simultaneously. This is due to the fact that either a peak lower than the lower limit or a saturated peak occurs.

The present disclosure has been made in view of the above-described issues and aims to provide a technique capable of accurately detecting a mutant gene and a mutation rate using capillary electrophoresis.

### Solution to Problem

A mutant gene detection method according to the present disclosure classifies signal peaks included in a detected signal into a first group that is lower than a first threshold and a second group that is not lower than the first threshold, increases an injection voltage until the signal peak belonging to the first group becomes higher than or equal to the first threshold, and reduces the injection voltage until the signal peak belonging to the second group becomes lower than or equal to a second threshold which is higher than the first threshold.

### Advantageous Effects of Invention

According to the mutant gene detection method according to the present disclosure, it is possible to accurately detect a mutant gene and a mutation rate using capillary electrophoresis. Other configurations, issues, advantages, and the like of the present disclosure will be clarified by the following description of embodiments.

### BRIEF DESCRIPTION OF DRAWINGS

FIG. 1 is a configuration diagram of an electrophoresis system 1 according to a first embodiment.

FIG. 2 illustrates signal peaks indicating results obtained by measuring a nucleic acid sample containing a mutant gene by the electrophoresis system 1.

FIG. 3 is an enlarged view of signal peaks of a probe group 202.

FIG. 4 illustrates results of measuring a DNA sample identical to the sample in FIG. 2 by increasing an injection voltage.

FIG. 5 illustrates a flowchart for explaining a general mutant gene detection method as a comparative example.

FIG. 6 illustrates a flowchart in a case where such a conventional technique as described in Patent Literature 1 is used for mutant gene detection as a comparative example.

FIG. 7 is a flowchart for explaining a mutant gene detection method according to the first embodiment.

### DESCRIPTION OF EMBODIMENTS

### first Embodiment: System Configuration

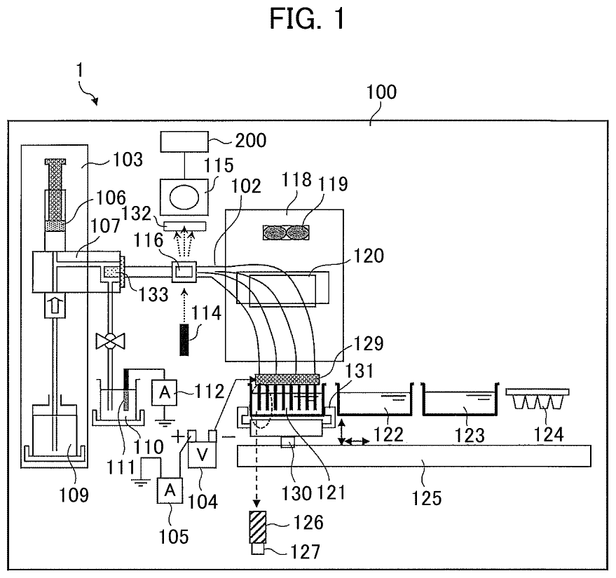

FIG. 1 is a configuration diagram of an electrophoresis system 1 according to a first embodiment of the present disclosure. The electrophoresis system 1 includes an electrophoresis apparatus 100 and a calculation apparatus 200 (computer). The electrophoresis apparatus 100 is an apparatus that analyzes a component contained in a sample by electrophoresisg the sample using a capillary.

The electrophoresis apparatus 100 includes a detection unit 116, a constant temperature bath 118, a conveyance unit 125, a high-voltage power source 104, a first ammeter 105, a second ammeter 112, the capillary 102, and a pump mechanism 103. The detection unit 116 optically detects a sample. The constant temperature bath 118 keeps the capillary 102 at a constant temperature. The conveyance unit 125 conveys various containers to capillary cathode ends. The high-voltage power source 104 applies a high voltage to the capillary 102. The first ammeter 105 measures an electrical current output by the high-voltage power source 104. The second ammeter 112 measures an electrical current flowing in an anode side electrode 111. The pump mechanism 103 injects a polymer into the capillary 102.

The capillary 102 is formed of a glass tube with an inner diameter of several tens to hundreds of microns and an outer diameter of several hundred microns, and has a surface coated with a polyimide to improve its strength. However, a polyimide coated film on a light irradiation portion which is irradiated with laser light is removed such that light emitted inside the portion easily leaks to the outside. The inside of the capillary 102 is filled with a separation medium to provide a difference in migration speed during electrophoresis. Although a fluid separation medium and a non-fluid separation medium are present, a fluid polymer is used as the separation medium in the first embodiment.

The detection unit 116 is a region of a portion of the capillary 102. When excitation light is emitted from a light source 114 to the detection unit 116, fluorescence (hereinafter referred to as information light) having a sample-dependent wavelength is generated from the sample and emitted outside the capillary 102. The information light is separated in a wavelength direction by a diffraction grating 132. An optical detector 115 analyzes the sample by detecting the separated information light.

The capillary cathode ends 127 are fixed through hollow electrodes 126 made of metal, and capillary tips protrude from the hollow electrodes 126 by approximately 0.5 mm. The hollow electrodes 126 disposed for each capillary are all integrally attached to a load header 129. All of the hollow electrodes 126 are electrically connected to the high-voltage power source 104 mounted on a main body of the apparatus. The hollow electrodes 126 operate as cathode electrodes when a voltage needs to be applied for electrophoresis, introduction of a sample, and the like.

Capillary ends (other ends) on the opposite side of the capillary cathode ends 127 are bundled together by a capillary head 133. The capillary head 133 is capable of being connected to a block 107 in a pressure-tight manner. The high voltage output by the high-voltage power source 104 is applied between the load header 129 and the capillary head 133. A syringe 106 fills the capillary with a new polymer from the other ends. Polymer refilling in the capillary is performed after each of measurements in order to improve the performance of the measurements.

The pump mechanism 103 includes the syringe 106 and a mechanism system for pressurizing the syringe 106. The block 107 is a connection member for communicating the syringe 106, the capillary 102, an anode buffer container 110, and a polymer container 109 with each other.

An optical detection unit that detects the information light from the sample includes the light source 114, the optical detector 115 for detecting light emitted in the detection unit 116, and the diffraction grating 132. To detect the sample separated by electrophoresis and present in the capillary, the light source 114 irradiates the detection unit 116 of the capillary, the diffraction grating 132 separates the emitted light from the detection unit 116, and the optical detector 115 detects the separated information light.

The constant temperature bath 118 is covered with a heat insulation material to keep the inside at a constant temperature, and the temperature is controlled by a heating and cooling mechanism 120. A fan 119 circulates and stirs the air in the constant temperature bath 118 to keep the temperature of the capillary 102 uniform and constant in terms of position.

The conveyance unit 125 includes up to three electric motors and a linear actuator and is movable along up to three axes in vertical, horizontal, and depth directions. At least one or more containers can be placed on a stage 130 of the conveyance unit 125. The stage 130 is provided with an electric grip 131, and a user can grasp and release each container via the grip 131. Therefore, a buffer container 121, a cleaning container 122, a waste liquid container 123, and a sample container 124 can be conveyed to the capillary cathode ends 127 as necessary. An unnecessary container is stored in a designated storage area in the apparatus.

The calculation apparatus 200 acquires a result of detecting the information light from the optical detector 115, analyzes the result of the detection to create a fluorescence strength waveform, and performs processing such as calculating a base length of a substance to be measured. Details of the processing performed by the calculation apparatus 200 will be described later. The calculation apparatus 200 can be configured by a central processing unit (CPU), software to be executed by the CPU, and the like, but can be configured by hardware such as a circuit device in which similar functions are implemented.

### First Embodiment: Issues of Conventional Techniques

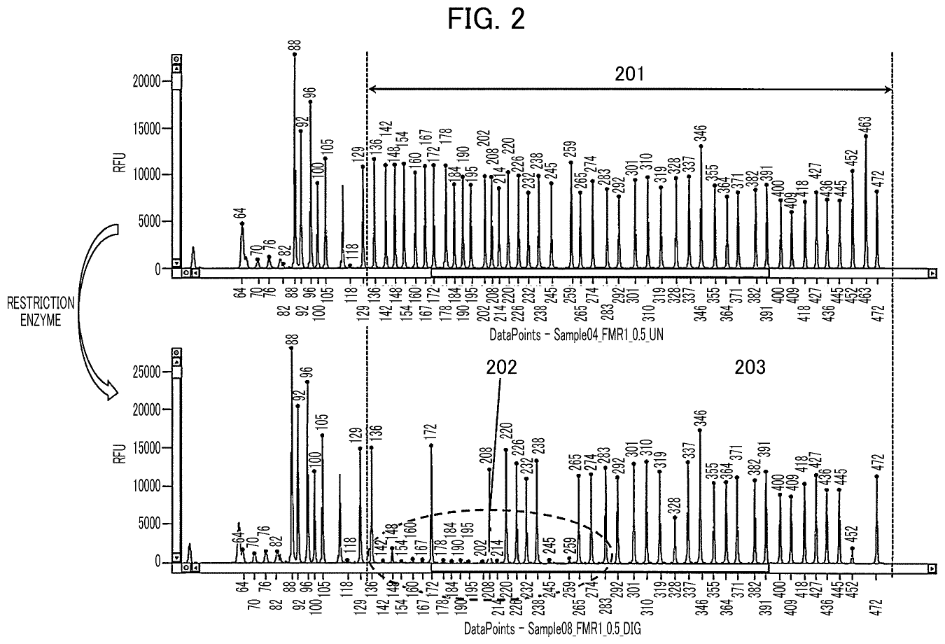

FIG. 2 illustrates signal peaks indicating results obtained by measuring a nucleic acid sample containing a mutant gene by the electrophoresis system 1. The purpose of the measurement is to calculate a mutation rate (methylation rate) of a DNA. In FIG. 2, 201 denotes a probe group that measures a methylation rate. 202 denotes a probe group that is cleaved by a restriction enzyme. 203 denotes a reference probe group that is not cleaved by the restriction enzyme. It can be seen that signal peaks of the probe group 202 are significantly lower than those of the probe group 203. This is due to the fact that the amount of the mutant gene is very small and a detected signal level is very low compared to a normal gene.

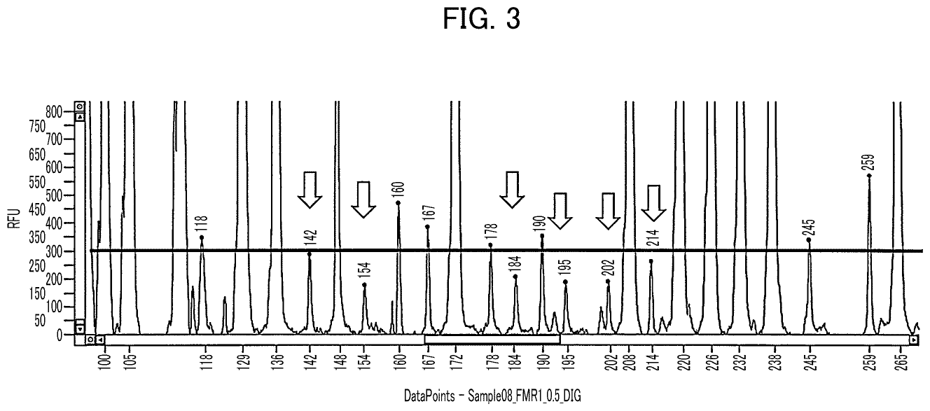

FIG. 3 is an enlarged view of the signal peaks of the probe group 202. Since a detected signal with a very low signal level has low reliability, it is common to exclude the detected signal from being analyzed. For example, when a signal level (vertical axis) 300 illustrated in FIG. 3 is set as an analyzable lower limit, 5 probes among 16 probes included in the probe group 202 and to be detected fall below the analyzable lower limit. Therefore, it is difficult to accurately calculate a mutation rate of this DNA sample.

The analyzable lower limit for signal peak levels can be determined based on whether the calculation apparatus 200 can acquire sufficiently reliable detected signal data. For example, if it is known that a detected signal below a certain signal level has a large amount of noise and low reliability, the signal level is set as the analyzable lower limit. This reliability varies depending on the type (for example, a product model number) of the electrophoresis apparatus 100, and thus it is possible to determine an analyzable lower limit for each type of electrophoresis apparatus 100.

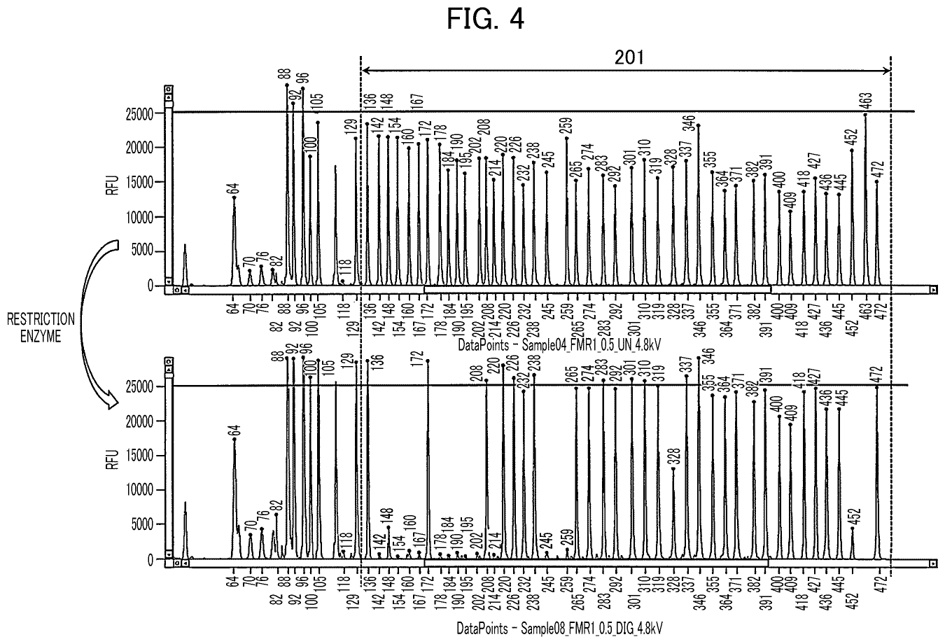

FIG. 4 illustrates results of measuring a DNA sample identical to the sample in FIG. 2 by increasing an injection voltage. It is considered that if a signal peak below the analyzable lower limit is present, a signal level is increased such that the signal peak becomes higher than or equal to the analyzable lower limit. For example, the overall signal level can be increased by increasing the injection voltage to be applied to the capillary when electrophoresis is performed. FIG. 4 illustrates the results.

The probe group 202 exhibits higher signal peaks than those in FIGS. 2 and 3 by increasing the injection voltage. However, a probe that exhibits a saturated signal peak of a normal gene is present (25000 is exceeded in the vertical axis in FIG. 4). Therefore, in this case, it is difficult to accurately calculate the mutation rate. This is due to the fact that signal levels (of saturated signal peaks) of some of normal genes cannot be accurately measured.

In view of the above description, in a mutant gene detection method according to the present disclosure, after the injection voltage is increased to the extent that a signal peak of the mutant gene can be analyzed, the injection voltage is reduced to the extent that other signal peaks are not saturated. Therefore, it is considered that the mutation rate can be accurately calculated.

### First Embodiment: Mutant Gene Detection Method

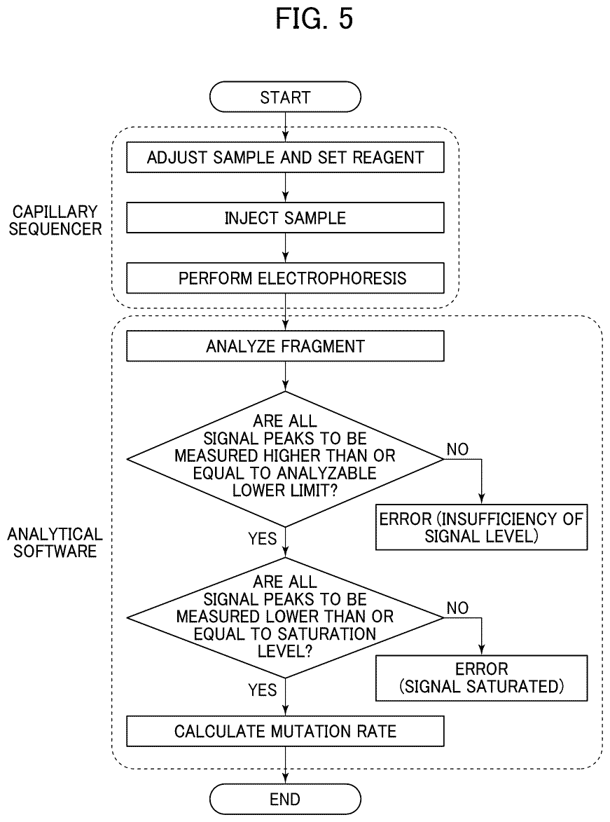

FIG. 5 illustrates a flowchart for explaining a general mutant gene detection method as a comparative example. In this method, first, electrophoresis is performed on a DNA sample using a capillary sequencer (such an electrophoresis system as illustrated in FIG. 1), and a detected signal obtained as a result of the electrophoresis is analyzed by software. If all of signal peaks to be measured are not higher than or equal to the analyzable lower limit, an error (analysis impossible) occurs because a signal level is insufficient. If all of the signal peaks to be measured are not lower than or equal to a saturation level, an error similarly occurs. If both of these conditions are satisfied, a ratio (mutation rate) of a signal peak of the mutant gene is calculated.

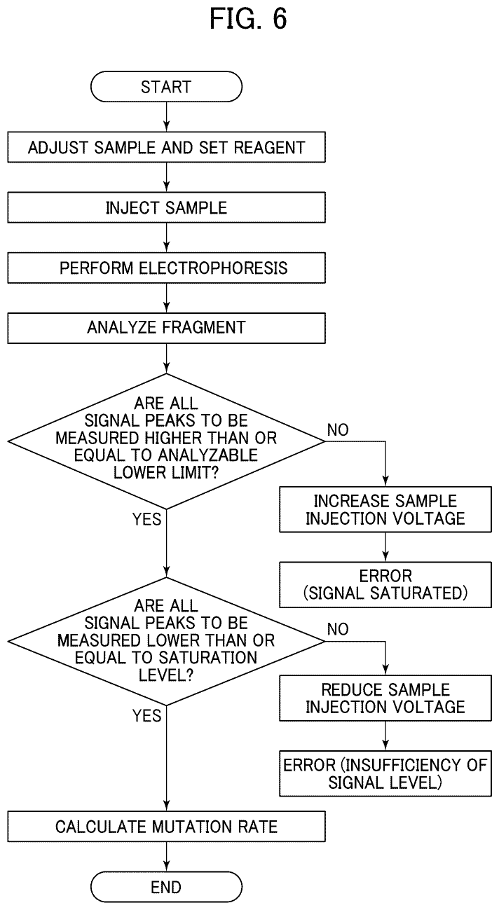

FIG. 6 illustrates a flowchart in a case where such a conventional technique as described in Patent Literature 1 is used for mutant gene detection as a comparative example. If all of signal peaks to be measured are not higher than or equal to the analyzable lower limit, a sample injection voltage is increased to increase the signal peaks to a level higher than or equal to the lower limit. However, if any signal level among signal peaks of normal genes is saturated, an error occurs. On the other hand, if all of the signal peaks to be measured are not lower than or equal to a saturation level, the sample injection voltage is reduced to reduce the signal peaks to a level lower than or equal to the saturation level. However, a signal peak of the mutant gene becomes lower than the analyzable lower limit and an error occurs. Therefore, in the conventional detection method, it is difficult to accurately calculate the mutation rate.

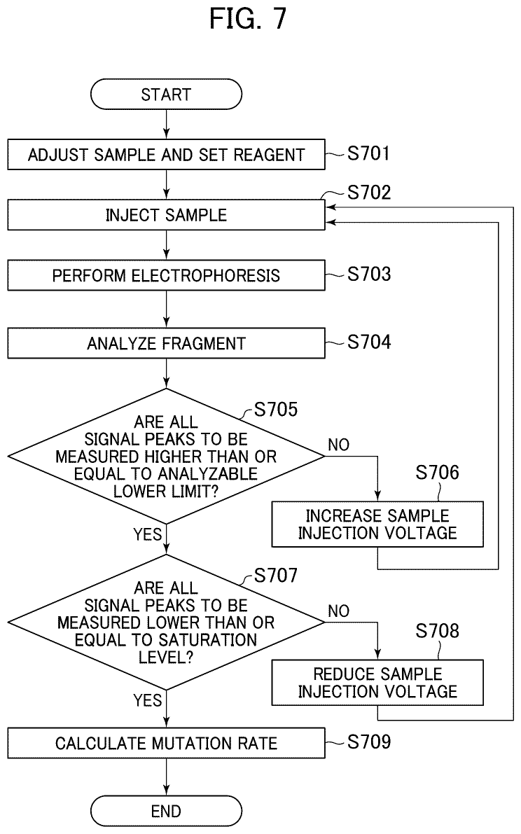

FIG. 7 is a flowchart for explaining a mutant gene detection method according to the first embodiment. This flowchart may be executed by a manual operation by an experimenter or may be executed by the calculation apparatus 200 controlling the electrophoresis system 1. It is assumed that the calculation apparatus 200 executes this flowchart in the following description, and each of steps illustrated in FIG. 7 is described below.

### (FIG. 7: Steps S701 to S703)

A user adjusts a DNA sample (nucleic acid sample) and sets a necessary reagent and the like (S701). The sample is introduced into the electrophoresis system 1 (S702) and electrophoresis is performed (S703).

### (FIG. 7: Steps S704 to S706)

The calculation apparatus 200 analyzes a detected signal of a fragment obtained by the electrophoresis (S704). If all of detected signal peaks to be measured are higher than or equal to the analyzable lower limit, the process skips to S707 (S705: YES). If a detected signal peak that is lower than the analyzable lower limit is present (S705: NO), the sample injection voltage (voltage to be applied to the capillary when the electrophoresis is performed) of the electrophoresis system 1 is increased (S706). The amount of the increase in this case may be determined in advance or may be determined based on the difference between the signal peak and the analyzable lower limit. At least until all of signal peak levels of a signal peak group derived from a mutant type become higher than or equal to the analyzable lower limit, S706 needs to be performed. After S706, the process returns to S702 and the electrophoresis is performed again using the increased injection voltage.

### (FIG. 7: Step S705: Supplement 1)

Among the signal peaks, a signal peak derived from the mutant type and a signal peak derived from a wild type are known in advance. Therefore, the information of the signal peak derived from either the mutant type or the wild type should be described as attribute information in advance, and each signal peak group derived from either the mutant type or the wild type can be identified by referencing the attribute data by the calculation apparatus 200. The same applies to S707.

### (FIG. 7: Step S705: Supplement 2)

When a signal peak is equal to the analyzable lower limit, the effect of noise is significant and the reliability of the signal is low. Since the reliability of the signal is generally determined by the type of the electrophoresis apparatus 100 (the electrophoresis system 1), the analyzable lower limit for signal peaks may be determined by each type of electrophoresis apparatus 100. Therefore, the calculation apparatus 200 may acquire the type of the electrophoresis apparatus 100 and set an analyzable lower limit level corresponding to the type. In other words, when a signal peak is lower than a certain lower limit threshold, and if the calculation apparatus 200 cannot accurately identify a mutant gene corresponding to the signal peak, the lower limit threshold may be set as the analyzable lower limit.

### (FIG. 7: Step S706: Supplement)

In this step, after the injection voltage is increased, the sample previously used is reused (remeasured) to execute S702 and the subsequent steps again. Therefore, S702 to S706 are executed using the same sample, and thus it is possible to suppress a measurement error caused by a difference between samples or the like. The same applies to a case where the process returns from S708 to S702.

### (FIG. 7: Steps S707 and S708)

If all of the detected signal peaks to be measured are lower than or equal to the saturation level, the process skips to S709 (S707: YES). If a detected signal peak higher than the saturation level is present (S707: NO), the sample injection voltage of the electrophoresis system 1 is reduced (S708). The amount of the reduction in this case may be determined in advance or may be determined based on the difference between the signal peak and the saturation level. At least until all of signal peak levels of a signal peak group derived from the wild type become lower than or equal to the saturation level, S708 needs to be executed.

After S708, the process returns to S702 and the electrophoresis is performed again using the reduced injection voltage.

### (FIG. 7: Steps S705 and S707: Supplement)

Only when “YES” is determined in each of these steps, the process proceeds to S709. In other words, the injection voltage is repeatedly adjusted such that the signal peaks fall within a range higher than or equal to the analyzable lower limit and lower than or equal to the saturation level by repeatedly executing S705 to S708 using the same sample. After this adjustment is completed, the signal peak derived from the wild type and the signal peak derived from the mutant type can be simultaneously measured by performing the next electrophoresis once.

### (FIG. 7: Step S707: Supplement)

The saturation level used in S707 may be determined based on the type of the electrophoresis apparatus 100 similarly to S705. That is, when an upper limit threshold with which the electrophoresis apparatus 100 and the calculation apparatus 200 can perform processing is present, the upper limit threshold may be determined as the saturation level. For example, as described later, when the mutation rate is calculated using a ratio of signal peak levels, and the signal peak derived from the wild type reaches the saturation level, the mutation rate cannot be accurately calculated. This is due to the fact that the original signal peak level is higher than the saturation level. Therefore, in this case, an upper limit signal level that can be output by the electrophoresis apparatus 100 is used as the saturation level in this step.

### (FIG. 7: Step S709)

The calculation apparatus 200 calculates the mutation rate of the DNA sample by using a result of fragment analysis to identify a ratio between a normal gene and the mutant gene. Signal peak levels of normal genes are substantially the same, and signal peak levels of mutant genes are substantially the same. Therefore, it is possible to calculate the mutation rate based on a ratio between a signal peak level of the normal gene and a signal peak level of the mutant gene.

### First Embodiment: Conclusion

The electrophoresis system 1 according to the first embodiment acquires, in advance, information indicating whether signal peaks obtained by performing the capillary electrophoresis on the DNA sample are derived from the mutant type or the wild type, and classifies the signal peaks into the derivation groups in accordance with the information. For the mutant type derivation group, the injection voltage is increased such that all of signal peaks become higher than or equal to the analyzable lower limit. For the wild type derivation group, the injection voltage is reduced such that all of signal peaks become lower than or equal to the saturation level. Therefore, both of the signal peaks derived from the mutant type and the signal peaks derived from the wild type can be measured by performing the electrophoresis once.

### Second Embodiment

The first embodiment describes that the detected signal peaks obtained by the electrophoresis are classified into the two groups, the mutant gene group that may be lower than the analyzable lower limit and the normal gene group that may be higher than the saturation level. The detected signal peaks can be divided into three or more groups. For example, when the sample contains a fragment in which signal strength of A among four bases of ATGC of a gene is relatively higher than signal strength of TGC, a signal peak corresponding to the fragment may be classified into a third group. Meanwhile, a fragment in which a signal peak is relatively low may be classified into a fourth group.

Although this classification is not classification into the mutant type and the wild type, (a) a relatively high signal peak group may be higher than the saturation level as in a wild type signal peak and thus the same processing as that for the wild type is required, and (b) a signal peak that belongs to a relatively low signal peak group and is lower than the analyzable lower limit is required to be subjected to the same processing as that for the mutant type. Therefore, in addition to the classification into the mutant type and the wild type, the signal peaks can be classified based on whether the signal peaks are higher than the saturation level and lower than the analyzable lower limit. The above-described classification of the signal peaks can be used in addition to or instead of the classification into the mutant type and the wild type. Therefore, the signal peaks can be classified into three or more groups.

When groups are configured based on whether the signal peaks are higher than the saturation level and lower than the analyzable lower limit, a range within which a signal peak level of each of the groups is identified in advance, and the information is described in the attribute data to be used in S705 (S707). That is, the information identifying whether the signal peaks that are lower than the analyzable lower limit and higher than the saturation level for each of the signal peaks is described in the attribute data. In S705, whether all of the groups are higher than or equal to the analyzable lower limit is determined. In S707, whether all of the groups are lower than or equal to the saturation level is determined. Therefore, the flowchart of FIG. 7 can be used as it is.

### Regarding Modifications of Present Disclosure

The present disclosure is not limited to the embodiments described above, and includes various modifications. For example, the embodiments are described above in detail in order to explain the present disclosure in an easy-to-understand manner, and are not necessarily limited to including all of the configurations described. In addition, some of configurations described in a certain embodiment can be replaced with a configuration described in the other embodiment. Further, to a configuration described in a certain embodiment, a configuration described in the other embodiment can be added. In addition, a configuration can be added to, removed from, or replaced with some of the configurations described in each of the embodiments.

In the embodiments described above, S706 is executed to increase the detected signal peaks, and thus if a similar effect can be obtained by alternative means other than increasing the injection voltage, the alternative means may be used. For example, the amount (concentration) of the sample to be introduced into the capillary of the electrophoresis apparatus 100 may be increased. The amount of the sample may be increased in combination with increasing the injection voltage. Similarly, in S708, the amount of the sample to be introduced into the capillary of the electrophoresis apparatus 100 may be reduced, or the amount of the sample to be introduced into the capillary of the electrophoresis apparatus 100 may be reduced in combination with reducing the injection voltage.

In the embodiments, the calculation apparatus 200 is described as the constituent component of the electrophoresis system 1, but the calculation apparatus 200 may be configured as a constituent component of the electrophoresis apparatus 100 to control each component of the electrophoresis apparatus 100.

### LIST OF REFERENCE SIGNS

1: Electrophoresis system
100: Electrophoresis apparatus
200: Calculation apparatus

1. A mutant gene detection method of detecting a mutant gene in a nucleic acid sample containing a gene, the method comprising:
a step of acquiring a detected signal by measuring the nucleic acid sample using a capillary electrophoresis apparatus;
a step of acquiring attribute information describing information indicating whether signal peaks included in the detected signal are lower than a first threshold;
in accordance with the attribute information, a step of classifying the signal peaks included in the detected signal into a first group that is lower than the first threshold and a second group that is not lower than the first threshold;
a step of increasing a voltage applied to a capillary in order for the capillary electrophoresis apparatus to perform electrophoresis on the nucleic acid sample until the signal peak belonging to the first group becomes higher than or equal to the first threshold; and
a step of reducing the voltage until the signal peak belonging to the second group becomes lower than or equal to a second threshold which is higher than the first threshold.

2. The mutant gene detection method according to claim 1, wherein
the attribute information describes information indicating whether the signal peaks included in the detected signal are derived from a mutant type or are derived from a wild type,
in the step of classifying, the signal peaks included in the detected signal are classified into the first group and the second group in accordance with the attribute information,
the first group is a group derived from the mutant type, and the second group is a group derived from the wild type.

3. The mutant gene detection method according to claim 1, wherein
the capillary electrophoresis apparatus includes a calculation apparatus that processes the detected signal,
the calculation apparatus sets the first threshold based on a type of the capillary electrophoresis apparatus,
the first threshold is higher than or equal to a lower limit signal level with which the calculation apparatus is capable of identifying the mutant gene, and
in the step of increasing the voltage, the voltage is increased until all of the signal peaks belonging to the first group become higher than or equal to the first threshold.

4. The mutant gene detection method according to claim 1, wherein
the capillary electrophoresis apparatus includes a calculation apparatus that processes the detected signal,
the calculation apparatus sets the second threshold based on a type of the capillary electrophoresis apparatus,
the second threshold is lower than or equal to an upper limit signal level with which the calculation apparatus is capable of analyzing the detected signal, and
in the step of reducing the voltage, all of the signal peaks belonging to the second group become lower than or equal to the second threshold.

5. The mutant gene detection method according to claim 1, further comprising:
after the step of increasing the voltage, a step of reacquiring the detected signal by remeasuring the nucleic acid sample using the capillary electrophoresis apparatus; and
a step of executing the step of increasing the voltage for the reacquired detected signal again.

6. The mutant gene detection method according to claim 1, further comprising:
after the step of reducing the voltage, a step of reacquiring the detected signal by remeasuring the nucleic acid sample using the capillary electrophoresis apparatus; and
a step of executing the step of reducing the voltage for the reacquired detected signal again.

7. The mutant gene detection method according to claim 1, further comprising:
a step of calculating a mutation rate of the nucleic acid sample, wherein
the mutant gene detection method executes the step of calculating the mutation rate when the signal peak belonging to the first group is higher than or equal to the first threshold and the signal peak belonging to the second group is lower than or equal to the second threshold.

8. The mutant gene detection method according to claim 7, wherein
the mutant gene detection method executes the step of increasing the voltage or the step of reducing the voltage without executing the step of calculating the mutation rate when at least either the signal peak belonging to the first group is lower than the first threshold or the signal peak belonging to the second group is higher than the second threshold.

9. The mutant gene detection method according to claim 1, wherein
the attribute information describes information identifying whether the signal peaks are lower than the first threshold for each of the signal peaks, and
in the step of classifying the signal peaks, a signal peak included in the detected signal is classified into a third group different from the first group and the second group in accordance with the attribute information,
the mutant gene detection method further comprising:
when the signal peak belonging to the third group is lower than the first threshold, a step of increasing the voltage until the signal peak belonging to the third group becomes higher than or equal to the first threshold; and
when the signal peak belonging to the third group is higher than the second threshold, a step of reducing the voltage until the signal peak belonging to the third group becomes lower than or equal to the second threshold.

10. The mutant gene detection method according to claim 1, further comprising:
a step of calculating a mutation rate of the sample based on a ratio of a signal level of the signal peak belonging to the first group and a signal level of the signal peak belonging to the second group.

11. The mutant gene detection method according to claim 1, wherein
in the step of increasing the voltage, an amount of the nucleic acid sample to be introduced into the capillary electrophoresis apparatus is increased instead of or in combination with increasing the voltage, and
in the step of reducing the voltage, the amount of the nucleic acid sample to be introduced into the capillary electrophoresis apparatus is reduced instead of or in combination with reducing the voltage.

12. The mutant gene detection method according to claim 1, further comprising:
a step of treating the nucleic acid sample using a multiplex ligation-dependent probe amplification (MLPA) method, wherein
in the step of acquiring the detected signal, the detected signal is acquired by measuring the nucleic acid sample treated using the MLPA method.
